(19) United States
(12) Patent Application Publication
(10) Pub. No.: US 2012/0032084 A1
(43) Pub. Date: Feb. 9, 2012

(54) DRIVE WITH CURVED LINEAR INDUCTION MOTOR

(75) Inventors: Joshua Sapp, Chardon, OH (US); Rosemarie Sheridan, Mayfield Village, OH (US); Charles L. Cattell, JR., Jefferson, OH (US); Terrance Nemenz, Chesterland, OH (US); Shufang Dong, Mayfield Heights, OH (US); Ted A. Bremenour, Concord Township, OH (US)

(73) Assignee: KONINKLIJKE PHILIPS ELECTRONICS N. V., Eindhoven (NL)

(21) Appl. No.: 13/263,768

(22) PCT Filed: Mar. 9, 2010
(86) PCT No.: PCT/IB10/51015

§ 371 Date: Oct. 10, 2011

Related U.S. Application Data
(60) Provisional application No. 61/169,327, filed on Apr. 15, 2009.

(51) Int. Cl. G01T 1/166; A61B 6/03; H02K 15/02; H02K 41/025

(52) U.S. Cl. 250/360.1; 310/12.31; 29/598

(57) ABSTRACT

A curved linear induction motor direct drive is provided. The rotor of the motor drive is mechanically attached to a rotating frame which in turn holds other components. The rotor may comprise two layers, an aluminum ring to provide the principal magnetic interaction with the stator, and a steel ring to provide mechanical strength. Such a rotor ring may be manufactured with a compression fit.

The present application relates generally to the art of electric motor design, and in particular concerns a drive having a curved linear induction motor. It has general application where the motor is used to move driven elements along a curved path, such as a circle or a portion thereof. Such a motor is useful for example in the imaging arts, and particularly in a method and apparatus for computed tomography (CT) based imaging. It has application at least in such imaging, where the motor drives rotation of a radiation source, a radiation detector, or both a source and detector, along a circular path in order to image a patient or object. Similar imaging systems which might use a curved linear induction drive motor include other x-ray based imaging systems and nuclear medicine imaging systems such as PET and SPECT. Thus the motor will be described herein with particular reference to a CT imaging system, with the understanding that it has more general applicability.

In the particular context of a CT imaging scanner, an x-ray source and one or more x-ray detectors are mounted on or to a rotating frame within a scanner housing or gantry. A person or object being imaged is positioned within the gantry between the x-ray source and the one or more x-ray detectors, as they rotate along a curved path around the person or object. The person or object is typically placed on a support table which can move linearly in and out of an aperture in the gantry, so that the x-ray source and the one or more x-ray detectors may be positioned axially at desired locations in performing an imaging scan.

The rotating frame which holds the x-ray source and detectors is often driven by an electric motor. In CT imaging scanners with a detector array having sixty-four or less slices, the rotational inertia and diameter of the frame and associated mounted components are relatively small, so that the rotational speed is slow (typically about 180 or less revolutions per minute). Thus the drive for such scanners can be a rotary AC induction motor having an indirect belt drive connection to the rotating frame, or a direct drive AC permanent magnet ring motor with a primary coil mounted on the gantry stator and a secondary permanent magnet ring mounted to the rotating frame for compactness.

In more modern CT imaging scanners with a detector array having two-hundred fifty-six slices of detector array to improve the imaging quality, however, such drives may often be inadequate for several reasons. The detector array hardware is commensurately larger and therefore heavier, and that extra weight and inertia must be borne by the rotating frame and controlled by the electric drive. In addition, these more modern CT imaging scanners typically employ an x-ray source with a higher power level then previous scanners, thus increasing the weight of the x-ray source which also must be borne by the rotating frame and controlled by the drive. Further, the central bore opening of the scanner gantry in these more modern CT imaging scanners is desirably larger than in previous scanners, to accommodate bariatric patients and also to aid interventional studies and procedures. Higher rotational speeds and acceleration rates are also desirable, in order to improve patient imaging throughout. These factors impose tight design constraints in terms of geometry, performance and cost, which are difficult to meet with a rotary AC induction motor having an indirect belt drive connection to the rotating frame, or a direct drive AC permanent magnet ring motor.

According to one aspect of the present invention, a curved linear induction motor direct drive is provided. Such a curved linear induction motor drive is better suited to the requirements of modern imaging scanners than either the rotary AC induction motors having an indirect belt drive, or the direct drive AC permanent magnet ring motors, used in previous scanners. The curved linear induction drive motor also has more general application to drive elements along a curved path in any sort of apparatus.

According to another aspect of the present invention, the rotor of the curved linear induction drive motor comprises a ring mechanically coupled to the rotating frame and having two layers, an aluminum layer and a steel layer. In one form of this aspect of the invention, the aluminum layer is the inner layer of a rotor ring, and the steel layer is an outer layer of the rotor ring. In yet another preferred form, the aluminum layer is inserted into and at least partially held within the steel layer by a compression fit. The aluminum layer provides the rotational driving force to the rotor ring. It transfers this driving force to the steel layer, which is mechanically coupled to the rotating frame. One advantage available with this aspect of the invention is that the steel layer can act as a heat sink to the aluminum layer and thus help to dissipate heat from the aluminum layer. An additional potential benefit is that the steel layer can complete the magnetic circuit and help generate the magnetic forces which produce rotational torque.

According to yet another aspect of the present invention, a method for making a rotor ring for use in a curved linear induction motor drive is provided comprising a compression fit process.

Numerous additional advantages and benefits will become apparent to those of ordinary skill in the art upon reading the following detailed description of preferred embodiments. The invention may take form in various components and arrangements of components, and in various process operations and arrangements of process operations. The drawings are only for the purpose of illustrating preferred embodiments and are not to be construed as limiting the invention.

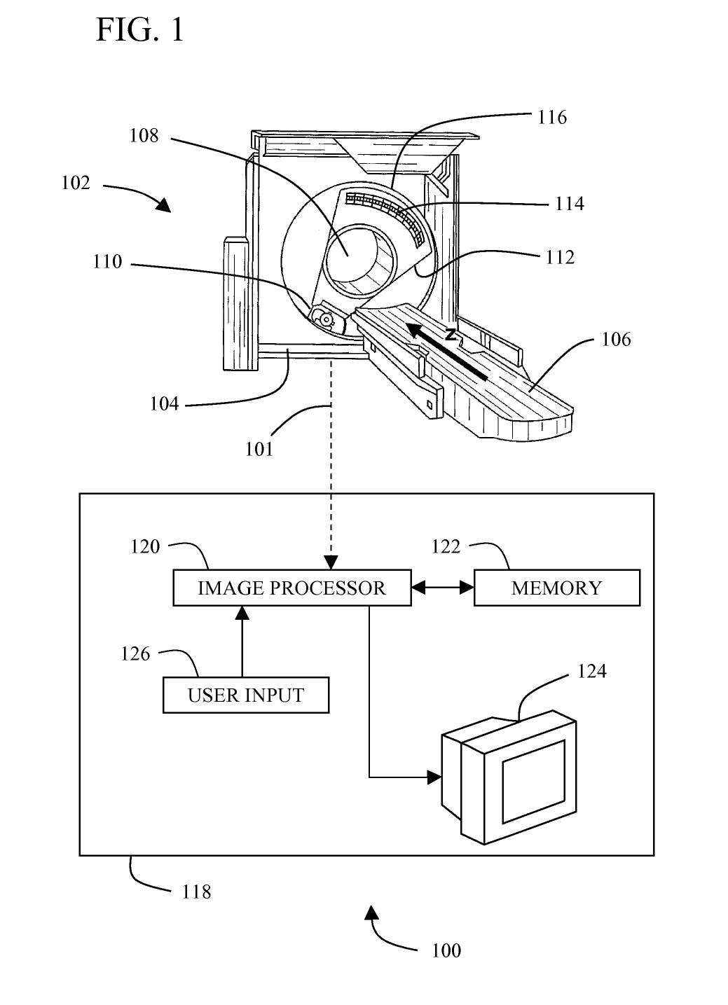

FIG. 1 illustrates a CT imaging scanner;

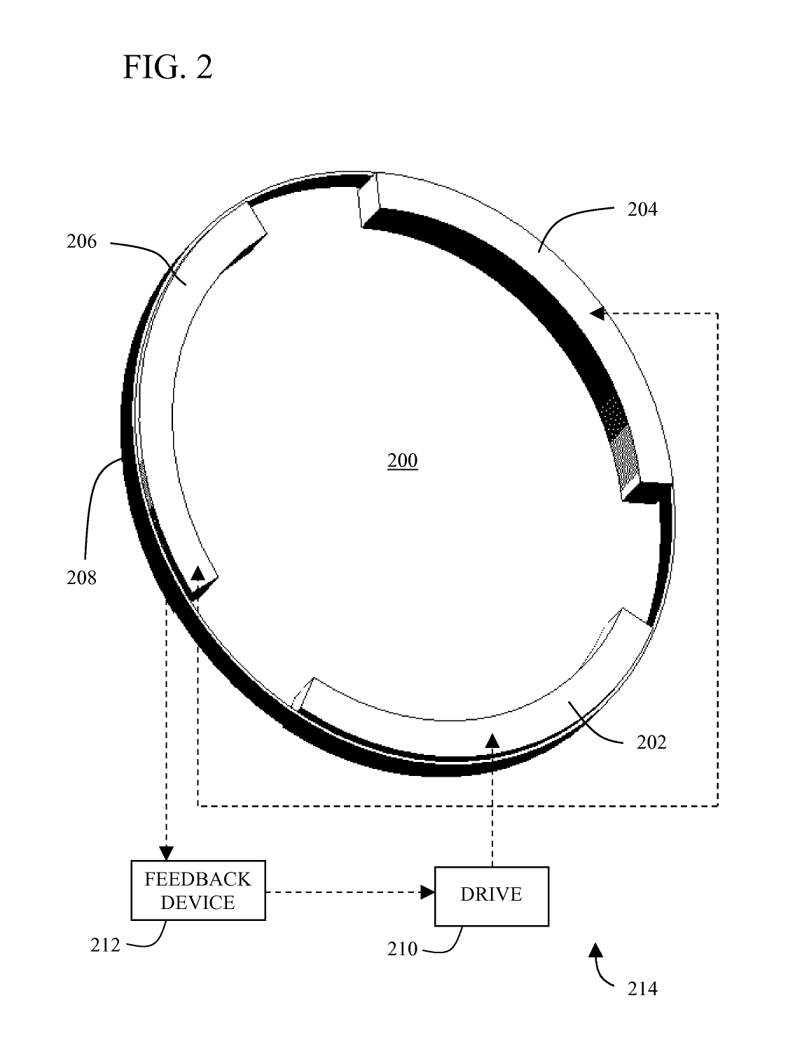

FIG. 2 illustrates a direct curved linear induction motor drive;

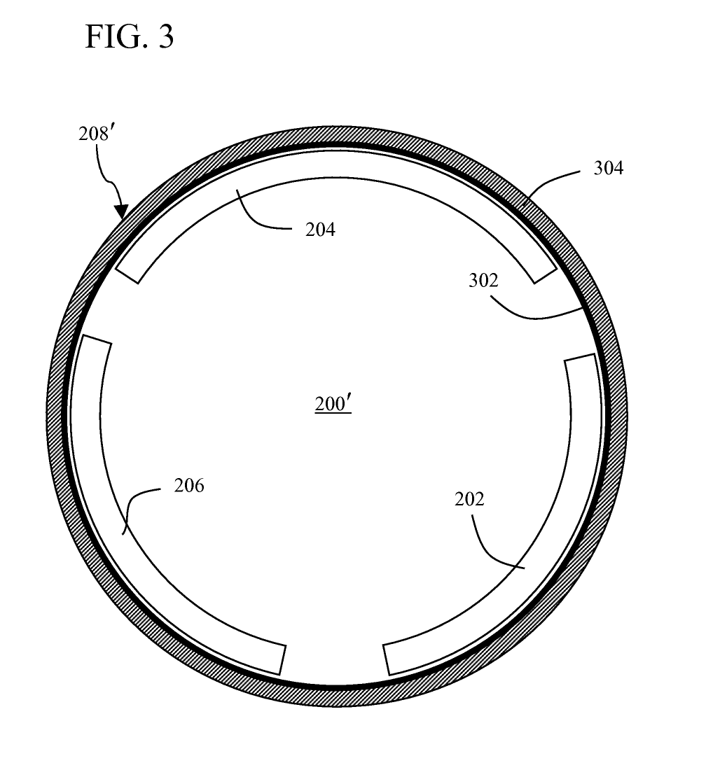

FIG. 3 illustrates a direct curved linear induction motor drive including a two layer rotor ring;

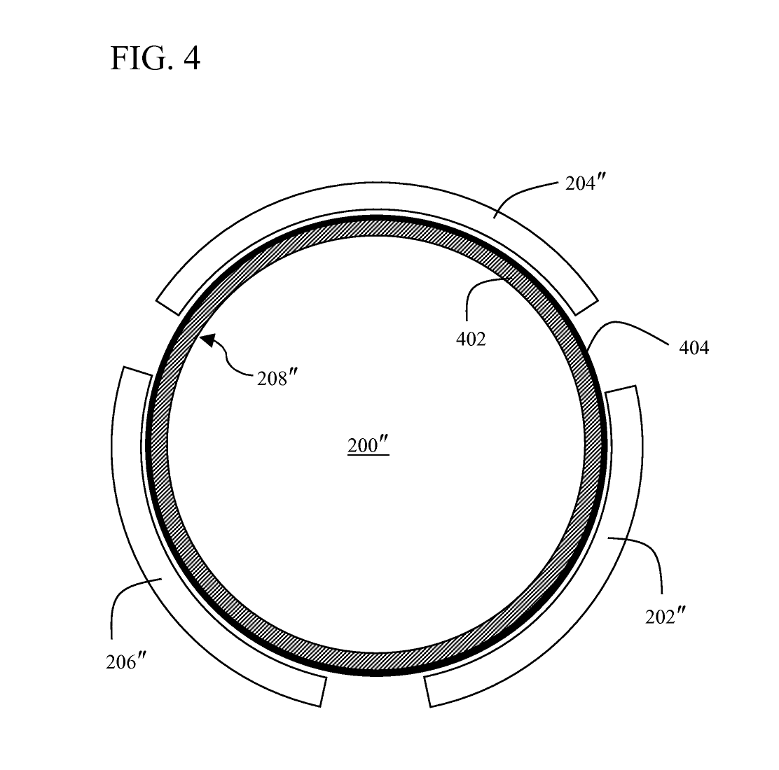

FIG. 4 illustrates an alternative direct curved linear induction motor drive including a two layer rotor ring;

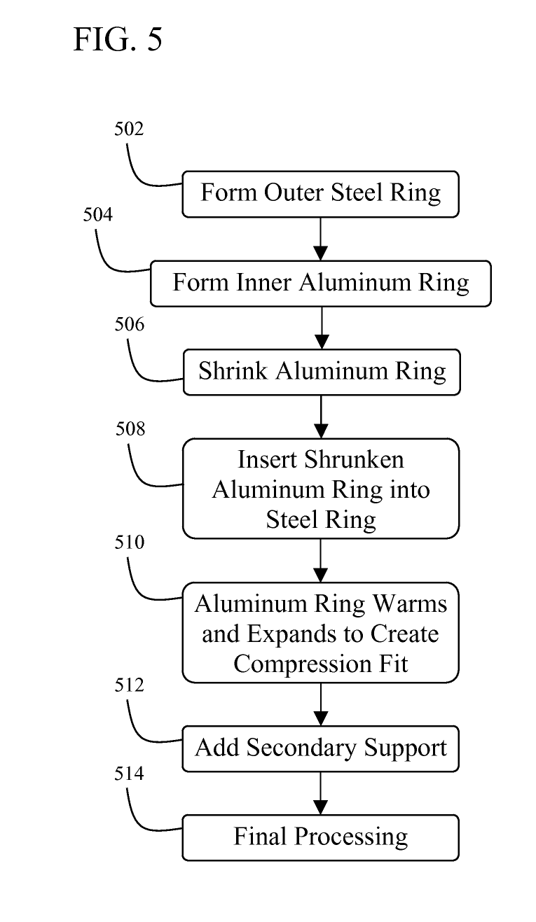

FIG. 5 illustrates a process of making a rotor ring with a compression fit between two layers; and

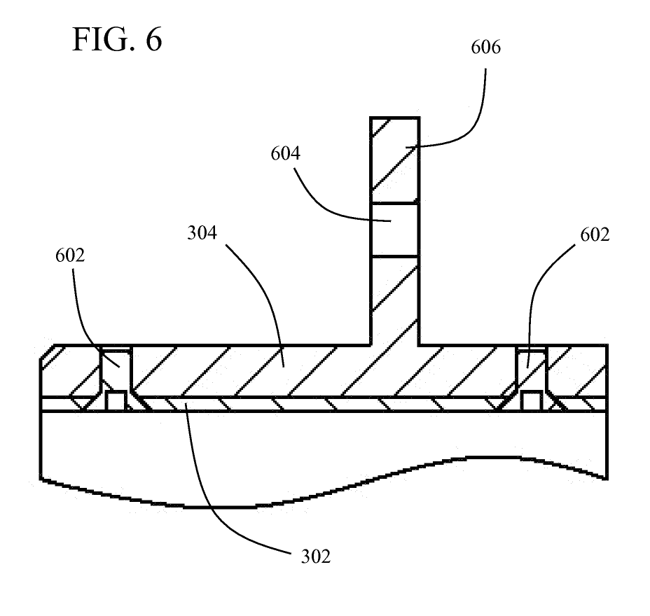

FIG. 6 illustrates a secondary support for a rotor ring having two layers assembled with a compression fit; and

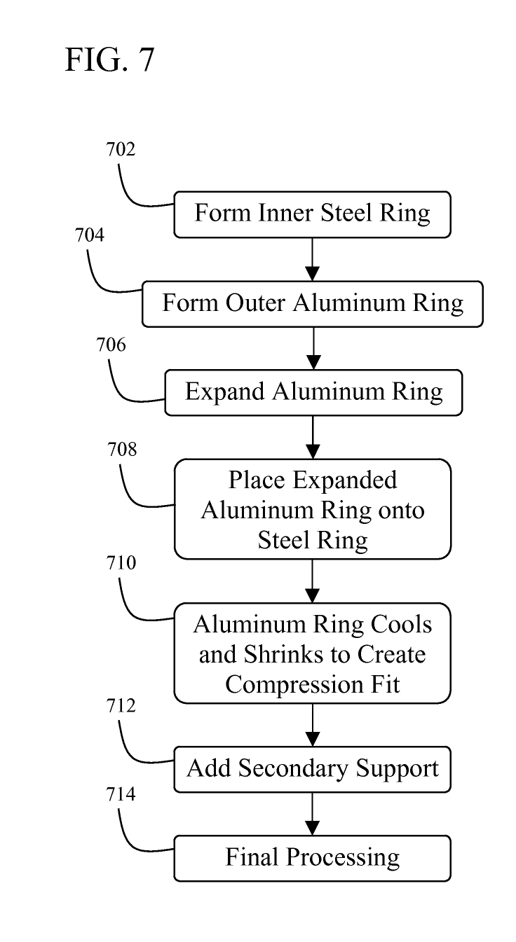

FIG. 7 illustrates a process of making a rotor ring with a compression fit between two layers.

The curved linear induction motor drive described here is directed generally to move driven elements along a curved path, such as a circle or a portion thereof, although it is described in the particular context of a CT imaging apparatus.

FIG. 1 illustrates one example of a CT imaging scanner 100 for performing an imaging scan. A CT imaging acquisition system 102 includes a gantry 104 and a table 106 which moves along the z-axis. A patient or other object to be imaged (not shown) lies down on the table 106 and is moved to be disposed within an aperture 108 in the gantry 104. Once the patient or object is in position, an x-ray source 110 emits a projection of x-rays 112 to be gathered by an x-ray detector array 114 inside the gantry 104. (A portion 116 of the gantry 104 is cut away in FIG. 1 to show the x-ray source 110 and x-ray detector array 114 which are housed inside the gantry 104.) The x-ray source 110 and x-ray detector array 114 rotate together around the aperture 108 to record CT imaging data from various positions, often in conjunction with linear movement of the table 106. This rotation is possible because the x-ray source 110 and detector array 114 are each mounted to a rotating frame (not shown) inside the gantry 104. The frame may be rotationally mounted in the gantry 104 in any manner, such as for example, using air bearings or steel roller bearings.

The CT imaging acquisition system 102 then passes the CT imaging data on to a CT imaging processing and display system 118 through a communication link 101. Although the systems 102 and 118 are shown and described here as being separate systems for purposes of illustration, they may in other embodiments be part of a single system. The CT imaging data passes to an image processor 120 which stores the data in a memory 122. The image processor 120 electronically processes the CT imaging data to generate images of the imaged patient or other object. The image processor 120 can show the resulting images on an associated display 124. A user input 126 such as a keyboard and/or mouse device may be provided for a user to control the processor 120.

As already mentioned, the x-ray source 110 and detector array 114 are each mounted to a rotating frame housed within the gantry 104. The rotation of the frame is driven by a direct curved linear induction motor drive. An exemplary such motor drive 200 is shown in FIG. 2. The motor 200 converts electric power to mechanical power to provide for the rotational positioning of the rotating frame, and therefore the x-ray source 110 and detector array 114 mounted on the frame, for CT scanning in a controllable manner. The illustrated exemplary direct segmented linear induction motor drive 200 includes three stator segments 202, 204 and 206, each comprising a curved linear induction motor primary coil pack. The curved stator segments are mounted within the gantry 104, and do not move. While three such curved stator segments are shown in FIG. 2, any number of such stator segments may be used, including only a single stator. Preferably two, three or four curved stator segments are used, and are placed symmetrically around the curved stator segments. In that way, the radial attractive forces between the curved stator segments and the rotor or rotor segments (described below) are balanced to cancel each other out.

The stator segments 202, 204, 206 are symmetrically placed within the circumference of a secondary rotor reaction ring 208. The rotor ring 208, in turn, is mechanically coupled to the rotating frame, although this is not shown in FIG. 2. In this way, the electrically driven rotation of the rotor ring 208 within the gantry 104 causes the frame to rotate as well. Each curved stator segment 202, 204, 206 of the motor 200 generates one third of the required thrust to propel or stop rotational movement of the rotor 208 around the stationary stator segments.

The rotor ring 208 is shown in FIG. 2 as a “full” ring, that is, it is one single segment which forms a complete and unbroken circle. In other, alternative embodiments, the ring 208 may be composed of more than one segment. The number of segments, and the extent of gaps between the segments, respectively making up the stator and the rotor of the motor are preferably chosen to avoid any “dead” positions of the rotor around the stator.

A conventional electronic servo drive or other drive 210 operates to vary the current, voltage or frequency of the electrical power applied to each of the stator segments 202, 204 and 206 to move the rotor 208, typically without need for commutation. Although alternating current is used to drive rotation, either alternating or direct current can be used to brake against rotation. A conventional feedback device 212 provides feedback from the rotor 208 to the drive 210, indicating the present rotational position of the rotor 208 (and therefore the x-ray source 110 and x-ray detector array 114 mounted to the rotor 208 via the frame). The motor 200, drive 210 and feedback device 212 together make up an entire rotational direct drive system 214.

In one embodiment shown in FIG. 3, a motor 200′ comprises the same curved stator segments 202, 204 and 206 of FIG. 2. In the motor 200′, the secondary rotor reaction ring 208′ is a full ring composed of two layers, an inner layer 302 and an outer layer 304. The inner layer 302 is made of a good electrical conductor such as for example aluminum, copper or silver. The inner layer 302 is preferably made of aluminum, and is relatively thin, on the order of about 2 millimeters in radial thickness. The outer layer 304 provides mechanical support and rigidity to the reaction ring 208′, and is preferably also composed of a magnetic material to complete the magnetic circuit and help generate the magnetic forces which produce rotational torque. Thus suitable materials for the outer layer 304 include iron or an iron alloy like steel, and particularly low carbon steel. The outer steel layer 304 is relatively thick, on the order of about 6 millimeters in radial thickness. The inner layer 302, the outer layer 304, or both, may be composed of multiple segments rather than the single segment forms shown in FIG. 3.

In connection with the embodiment of FIG. 3, where the inner layer 302 is aluminum and the outer layer 304 is steel, the curved stator segments 202, 204, 206 are composed of three identical segments of silicon steel lamination with copper coils around the lamination slots. The slotted stator coils face outward, toward the inner aluminum layer 302 of the rotor 208′, to induce electric current and rotate the rotor 208′. Thus the inner aluminum layer 302 provides the principal rotational driving force to the rotor 208′, in response to the magnetic interaction between the curved stators 202, 204 and 206 and the aluminum.

The outer steel layer 304, in turn, is mechanically coupled to the rotating frame (such as shown for example in FIG. 6). This mechanical coupling may be a direct coupling, where there are no intervening structural elements between the ring 208′ and the frame, or it may be indirect where there are intervening elements. One example of an indirect mechanical coupling occurs where the ring 304 is bolted to the race of an air bearing or a roller bearing, and the race in turn is fixedly attached to the frame.

In an alternative curved linear induction direct drive system motor 200″, shown in FIG. 4, the curved stator segments 202″, 204″ and 206″ can be situated at the outer side of the rotor 208″. In the motor 200″, the secondary rotor reaction ring 208″ is a full ring composed of two layers, an inner layer 402 and an outer layer 404. The inner layer 402 provides mechanical support and rigidity to the reaction ring 208″, and is preferably also composed of a magnetic material to complete the magnetic circuit and help generate the magnetic forces which produce rotational torque. Thus suitable materials for the inner layer 402 include iron or an iron alloy like steel, and particularly low carbon steel. The inner steel layer 402 is relatively thick, on the order of about 6 millimeters in radial thickness. The outer layer 404 is made of a good electrical conductor such as for example aluminum, copper or silver. The outer layer 404 is preferably made of aluminum, and is relatively thin, on the order of about 2 millimeters in radial thickness. The inner layer 402, the outer layer 404, or both, may be composed of multiple segments rather than the single segment forms shown in FIG. 3.

In connection with the embodiment of FIG. 4, where the inner layer 402 is steel and the outer layer 404 is aluminum, the curved stator segments 202″, 204″, 206″ are composed of three identical segments of silicon steel lamination with copper coils around the lamination slots. The slotted stator coils face inward, toward the outer aluminum layer 404 of the rotor 208″, to induce electric current and rotate the rotor 208″. Thus the outer aluminum layer 404 provides the principal rotational driving force to the rotor 208″, in response to the magnetic interaction between the curved stators 202″, 204″ and 206″ and the aluminum. The inner steel layer 402, in turn, is mechanically coupled to the rotating frame such as with bolts (not shown). The inner layer 402, the outer layer 404, or both, may be composed of multiple segments rather than the single segment forms shown in FIG. 4.

One advantage of the embodiments shown in FIGS. 3 and 4 is that the steel layer extracts heat from the aluminum layer, and also helps complete the magnetic circuit and generate the magnetic forces which produce the rotational torque. Because the steel layer is much larger than the aluminum layer, such heat transfer minimizes temperature deformations of either layer.

The rotor 208′ of FIG. 3 can be manufactured using a compression fit between the inner aluminum ring 302 and the outer steel ring 304. The primary benefit of providing such a compression fit is a substantially even distribution of stresses around the entire circumference of the rotor 208′, resulting in optimal performance of the motor 200′. The preload, or resultant residual stresses, of the aluminum ring 302 compressed within the steel ring 304 provides an even friction force which prevents relative motion between the two pieces 302 and 304. Due to the compression fit, the magnetic driving force that is generated by the curved linear induction motor 200′ in the aluminum ring 302 is transferred into the steel ring 304. In addition, the consistent pressure around the circumference of the rotor 208′ helps to prevent a build-up of load at a single point in the inner aluminum ring 302. Such load build-ups could cause failure of the aluminum ring 302, which is relatively thin in comparison to the outer steel ring 304. Another feature of the compression fit is that the compressive forces keep the inner aluminum ring 302 in contact with the outer steel ring 304 substantially throughout the circumference of the interface. This helps to aid the steel ring 304 act as a heat sink with respect to the aluminum ring 302.

Such a compression fit may be achieved, for example, by shrink fitting the aluminum ring 302 into the steel ring 304. FIG. 5 illustrates an exemplary process for achieving a compression fit of an inner aluminum ring 302 in an outer steel ring 304. In step 502, the steel ring 304 is formed such as by machining to have approximately the proper geometry for the application. In step 504, a substantially rectangular aluminum plate is rolled, and its ends welded together, to form the inner aluminum ring 302. The outer diameter of the inner aluminum ring 302 is somewhat larger than the inner diameter of the outer steel ring 304. The first two steps 502 and 504 of this process may be performed in any order. The inner aluminum ring 302 is placed in a cold atmosphere to cause it to shrink 506 in size. For example, the ring 302 may be placed in a liquid nitrogen bath or other substance which is sufficiently cold to cause the aluminum to shrink. The inner aluminum ring 302 remains in the cold atmosphere until the outside diameter of the inner aluminum ring 302 is smaller than the inside diameter of the outer steel ring 304. The shrunken inner aluminum ring 302 is then inserted 508 into the outer steel ring 304 and permitted to warm up, and thus expand. As the inner aluminum ring 302 expands 510, residual compressive stresses are formed between the two rings 302 and 304 resulting in frictional forces which hold the rings together. A secondary support such as fasteners or a bonding agent can be added 512 to strengthen the bond between the two rings 302, 304. The final assembly may be machined or otherwise processed 514 to achieve dimensional specifications for the particular application. Such final processing may include, for example, machining the inside diameter of the aluminum ring to match specified dimensions.

As mentioned, a secondary support can be applied 512, in addition to the compression fit, to hold the inner aluminum ring 302 within the outer steel ring 304. One such secondary support may include countersinking screws 602 into the inner aluminum ring 302 and outer steel ring 304, as shown in FIG. 6. Application of such screws 602 adds additional compressive forces between the inner aluminum ring 302 and the outer steel ring 304 to increase the friction force and make the connection more robust. As also shown in FIG. 6, the outer steel ring 304 may include a series of tabs 604 with apertures 606 therein for receiving a bolt to form a mechanical coupling of the ring 304 to a rotating frame, as discussed above.

Another type of secondary support involves applying a bonding agent between the inner aluminum ring 302 and the outer steel ring 304, that would cure once the two rings are held together with the compression fit. The bonding agent could either cure over time in a natural atmosphere, or cure anaerobically (without the presence of air). The bonding agent is preferably liquid at the cooled temperature of the aluminum ring 302, before it is inserted into the steel ring 304, so that the bonding agent is not adversely affected before the aluminum is completely warmed up. In addition, the bonding agent preferably does not materially impact heat transfer between the aluminum and the steel, thus allowing the substantially free flow of heat from the aluminum to the steel.

The embodiment of FIG. 4 may also be manufactured using a compression fit, between the inner steel ring 402 and the outer aluminum ring 404. Such a compression fit may be achieved, for example, by shrink fitting the outer aluminum ring 404 onto the inner steel ring 402. FIG. 7 illustrates such a process. Here, the inner steel ring 402 is formed 702 and the outer aluminum ring 404 is formed 704. The outer diameter of the inner steel ring 402 is somewhat larger than the inner diameter of the outer aluminum ring 404. The outer aluminum ring 404 is then placed in an oven or other heated atmosphere to cause it to expand in size 706, until its inner diameter is larger than the outside diameter of the inner steel ring 402. The expanded outer aluminum ring 404 is then placed 708 around the inner steel ring 402 and permitted to cool, and thus shrink 710. As the outer aluminum ring 404 shrinks, residual compressive stresses are formed between the two rings 402 and 404 resulting in frictional forces which hold the rings together. A secondary support such as fasteners or a bonding agent can be added 712 to strengthen the bond between the two rings 402, 404. The final assembly may be machined or otherwise processed to achieve dimensional specifications for the particular application. Such final processing may include, for example, machining the outer diameter of the aluminum ring to match specified dimensions.

Based on the specific design parameters of a given CT imaging scanner 100, such as thrust and rotational speed requirements, the design of the motor 200 and the selection of the drive 210 are considered together to minimize the required nominal power level of Volt-Amperes and reduce the volume taken up by the drive system 214 in the gantry 104, as well as its cost. The orientation and location of the rotor 208 and curved stator packs 202, 204 and 206 can be determined based on the available gantry space. In a preferred embodiment, the motor 200 may be a three phase induction motor, with three segmented stators connected serially or in parallel, with either a star scheme or a delta scheme.

The design of a segmented linear induction motor direct drive system 214 for the needs of a particular CT imaging apparatus may be optimized in the following manner. First, determine the size W of the electronic motor drive system 214 according to the required peak thrust level F, the peak linear speed ν, the efficiency of the motor η, and the power factor of the motor cos θ. The line voltage and current of the motor drive system 214 output are determined based on the size W. From that voltage and current, the phase current and voltage limit of each stator segment can be calculated. Finally, the design of the linear induction motor pack stators is chosen to provide the desired thrust output at the desired linear speed, or the equivalent excitation frequency with the proper slip frequency.

In a preferred embodiment, the size W is selected according to the following formula:

$$W = \frac{F \cdot v}{η \cdot cos θ} . \qquad ( Eq . 1 )$$

Once the desired peak line voltage V_{L} is determined, then the drive line current I_{L} can be calculated for a three phase drive according to the formula:

W=\sqrt{3}·V_{L}·I_{L} (Eq. 2).

The three phases of the motor can be connected with a delta scheme or a star scheme. As a representative example to demonstrate the design process, the following assumes the three phases of the motor are connected with a delta scheme. For three stator motor packs such as 202, 204, 206 connected in parallel, the phase voltage of each motor pack is the same as the drive line voltage V_{L}, and the phase current of the motor pack is determined from:

I=I_{L}/3\sqrt{3} (Eq. 3).

Thus, assume for example that the peak thrust F is 900 Newtons, the peak linear speed ν is 18.4 meters per second, the efficiency η of the linear motor is 48% or 0.48, and the power factor cos θ of the linear motor is 0.55 at peak speed. Applying Equation 1 to those system performance specifications, the drive size W is 62727 Volt-Amperes for peak output. If the peak line voltage V_{L} of the drive output is 460 Volts, then according to Equation 2, the peak line current I_{L} of the drive should be 78.7 Amps. For three motor packs 202, 204, 206 connected in parallel with a delta scheme, and applying Equation 3, the phase current of each motor symbol is 15.2 Amps, and the phase voltage of each motor pack is 460 Volts.

Once the phase current and the phase voltage of the linear induction motor packs are determined, the next step is to design the lamination and winding schemes of the motor packs to achieve the desired thrust level at the desired speed. In the representative example described above, each motor pack should achieve the 300 Newtons of thrust at the peak speed of 18.4 meters per second. This iterated motor design process attempts to maximize the thrust generation at peak speed through motor impedance matching, and fully utilize the calculated phase current and voltage. The details of designing a lamination and winding scheme design for the stator segments, once the design requirements and constraints are determined, will be well-known to a person of ordinary skill in this art.

To help prevent the linear induction motor from overheating, thermal sensors such as negative temperature coefficient (NTC) sensors may be embedded in all phases of the stators. Such sensors can be placed in the most likely hot spots of the motor, including near the center of the stator segments such as 202, 204 and 206. The thermal sensors are used to measure the temperature of the motors in real time so that power to the stator segments may be cut off if a pre-set critical temperature is reached, and thus prevent the motor segments from overheating. To achieve this, thermal switches may be located in close proximity to each thermal sensor, in order to cut off power to a potentially overheating motor segment. The thermal switches may be, for example, normally closed with a cut-off temperature of 150 degrees Celsius. The thermal switch will be activated to remove drive power when the thermal sensor reaches 150 degrees Celsius.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof. The invention may take form in various components and arrangements of components, and in various steps and arrangements of steps. The drawings are only for purposes of illustrating the preferred embodiments and are not to be construed as limiting the invention.

1. A curved linear induction motor drive system comprising a motor controlled by a drive, the motor comprising a stator and a rotor, the stator comprising one or more stator segments which are curved and are controlled by the drive, and the rotor comprising a ring mechanically coupled to a rotatable frame, wherein the rotor is magnetically driven by the one or more curved stator segments to rotate along a curved path.

2. The curved linear induction motor drive system of claim 1, wherein the ring is comprised of at least two ring segments separated by gaps between the ring segments.

3. The curved linear induction motor drive system of claim 1, wherein at least one of a radiation source and a radiation detector is mounted to the rotatable frame.

4. The curved linear induction motor drive system of claim 1, wherein the curved stator segments are disposed within a circumference of the ring, and an outer curve of the curved stator segments closely corresponds to an inner curve of the ring.

5. The curved linear induction motor drive system of claim 1, wherein the ring comprises an inner layer and an outer layer, one of the inner layer and the outer layer comprises an electrical conductor to be magnetically driven by the curved stator segments, and the other of the inner and the outer layers completes the magnetic circuit and provides mechanical support to the electrical conductor.

6. The curved linear induction motor drive system of claim 5, wherein the electrical conductor includes aluminum, and the other of the inner and the outer layers includes steel.

7. The curved linear induction motor drive system of claim 5, wherein the inner layer of the ring is the electrical conductor, and the outer layer of the ring completes the magnetic circuit and provides mechanical support to the inner layer.

8. The curved linear induction motor drive system of claim 5, wherein the inner layer of the ring is held within the outer layer by a compression fit between the inner and outer layers.

9. The curved linear induction motor drive system of claim 8, further comprising a secondary support between the inner and outer layers of the ring.

10. The curved linear induction motor drive system of claim 1, wherein there are three, four or five curved stator segments.

11. The curved linear induction motor drive system of claim 1, wherein the motor is a three phase induction motor having three curved stator segments, and the size W is determined from a peak thrust level F, a peak linear speed ν, an efficiency of the motor η, and a power factor of the motor cos θ, according to:
$$W = \frac{F \cdot v}{η \cdot cos θ} .$$

12. The curved linear induction motor drive system of claim 11, wherein a drive line current I_{L} is determined from the size W and a peak line voltage V_{L}, according to:
W=\sqrt{3}V_{L}·I_{L}.

13. The curved linear induction motor drive system of claim 1, disposed within an imaging apparatus.

14. A curved linear induction motor drive system comprising a drive and a motor controlled by the drive, the motor comprising a stator and a rotor, the stator comprising one or more stator segments which are curved and are controlled by the drive, and the rotor comprising an aluminum layer to be magnetically driven by the one or more curved stator segments to rotate along a curved path.

15. The curved linear induction motor drive system of claim 14, wherein the aluminum layer is disposed in a ring which is mechanically coupled to a rotatable frame.

16. The curved linear induction motor drive system of claim 14, wherein at least one of a radiation source and a radiation detector is coupled to the rotor.

17. A process of manufacturing a ring for use as a rotor of a curved linear induction motor drive system, wherein the ring comprises an aluminum layer and a steel layer, the process comprising:
forming the aluminum layer and the steel layer into ring shapes, wherein an outer diameter of the aluminum ring is larger than an inner diameter of the steel ring;
placing the aluminum ring in a cold atmosphere to cause it to shrink in size, until the outer diameter of the aluminum ring is smaller than the inner diameter of the steel ring;
inserting the aluminum ring into the steel ring; and
permitting the aluminum ring to increase in temperature, so that it expands within the steel ring.

18. The process of claim 17, further comprising adding a secondary support between the aluminum ring and the steel ring.
